(19) United States
(12) Patent Application Publication
(10) Pub. No.: US 2024/0115183 A1
(43) Pub. Date: Apr. 11, 2024

(54) Wireless Recording System-on-chip for Distributed Neural Interface Systems with Inductive Power Delivery and UWB Data Transmission

(71) Applicant: The Regents of the University of California, Oakland, CA (US)

(72) Inventors: Hamed Rahmani, Belle Mead, NJ (US); Aydin Babakhani, Los Angeles, CA (US)

(73) Assignee: The Regents of the University of California, Oakland, CA (US)

(21) Appl. No.: 18/257,997

(22) PCT Filed: Dec. 20, 2021
(86) PCT No.: PCT/US2021/073036

§ 371 Date: Jun. 16, 2023

Related U.S. Application Data
(60) Provisional application No. 63/127,702, filed on Dec. 18, 2020.

(51) Int. Cl. A61B 5/293; A61B 5/283; A61N 1/36; A61N 1/372; H02J 50/00; H02J 50/10; H02J 50/20; H02J 50/40

(52) U.S. Cl. CPC A61B 5/293; A61B 5/283; A61N 1/36125; A61N 1/37288; H02J 50/001; H02J 50/10; H02J 50/20; H02J 50/402; H02J 2310/23

(57) ABSTRACT

Systems and method for wireless recording system-on-chips for distributed neural interface systems with inductive power delivery and UWB data transmission are described. In an embodiment, the system includes an implantable neural interface including: an electrode array having several electrodes; front end circuitry including: one or more digital components, and at least one amplifier coupled to a first electrode and a second electrode of the electrode array, wherein the amplifier and the first electrode and the second electrode form a sensing channel configured to sense electrical activity; and a transceiver including: several digital components; a power harvesting system that receives RF energy through a wireless power link; and a wireless clock receiver that provides a clock signal to the one or more digital components of the front end circuitry and the several digital components of the transceiver.

### CROSS REFERENCE TO RELATED APPLICATIONS

This application is a national stage of PCT Patent Application No. PCT/US2021/073036 entitled “Wireless Recording System-on-chip for Distributed Neural Interface Systems with Inductive Power Delivery and UWB Data Transmission” filed Dec. 20, 2021, which claims priority to U.S. Provisional Patent Application Ser. No. 63/127,702, entitled “Wireless Recording System-on-chip for Distributed Neural Interface Systems with Inductive Power Delivery and UWB Data Transmission”, to Rahmani et al., filed on Dec. 18, 2020, the disclosures of which are herein incorporated by reference in their entirety.

### FIELD OF THE INVENTION

The present invention generally relates to wireless recording system-on-chips for distributed neural interface systems with inductive power delivery and ultrawideband (UWB) data transmission.

### BACKGROUND

Emerging applications of biomedical devices have shown an ever-increasing demand for data acquisition with high bandwidth and higher resolutions. For instance, research on the anatomical, physiological, and computational bases of human cognitive and motor functions has made important strides in recent years, yet has been limited by a lack of information on the dynamics of processes. This can be a methodological limitation related to the low spatial and temporal resolution of widely available tools such as fMRI, EEG, behavioral, and stroke lesion-based approaches. Today, neural interface implantable systems are becoming increasingly popular as they have demonstrated great potentials in novel diagnostic and treatment methods. They can be used in a variety of applications such as brain-machine interface (BMI) systems, cochlear implants, and retinal prosthesis, among various other applications. To address clinical constraints and alleviate infection risks, wireless operation is a necessity for human implantable systems.

### SUMMARY OF THE INVENTION

Systems and method for wireless recording system-on-chips for distributed neural interface systems with inductive power delivery and UWB data transmission are described. In an embodiment, the system includes an implantable neural interface including: an electrode array having several electrodes; front end circuitry including: one or more digital components, and at least one amplifier coupled to a first electrode and a second electrode of the electrode array, wherein the amplifier and the first electrode and the second electrode form a sensing channel configured to sense electrical activity; and a transceiver including: several digital components; a power harvesting system that receives RF energy through a wireless power link; and a wireless clock receiver that provides a clock signal to the one or more digital components of the front end circuitry and the several digital components of the transceiver.

In a further embodiment, the at least one amplifier is configured to increase a voltage amplitude of a signal corresponding to the sensed electrical activity.

In a further embodiment, the one or more digital components of the front end circuitry include an Analog to Digital Converter (ADC) coupled to the at least one amplifier and configured to convert an amplified signal to a digital signal.

In a further embodiment, the transceiver includes a data transmitter (TX) configured to transmit data corresponding to the sensed electrical activity; and the one or more digital components of the front end circuitry include a Parallel-Input-Serial-Output (PISO) shift register unit coupled to receive the digital signal from the ADC and configured to output digitized data corresponding to the digital signal to the transceiver.

In a further embodiment, the transceiver further includes: a data receiver (RX) for receiving data from an external reader on a wireless downlink (DL); a reconfigurable data transmitter (TX) for transmitting data to the external reader on a wireless uplink (UL); several antennas that enable simultaneous power delivery and data communication through two distinct wireless links separated in the frequency domain; and a receiver antenna that is shared between the power harvesting system and the RX.

In a further embodiment, the data TX includes a power oscillator (PO) directly connected to a transmitter antenna for wireless data transmission.

In a further embodiment, DL data is incorporated into the wireless power link with an amplitude-shift-keying (ASK) modulation scheme.

In a further embodiment, simultaneous UL and DL communication is enabled using frequency division duplexing (FDD), wherein a center frequency of the UL is in the GHz region.

In a further embodiment, the data TX is configured to transmit UL data with either on-off-keying (OOK) and ultrawideband (UWB) modulation.

In a further embodiment, the RX is directly powered by the power harvesting system and is active during operation of the neural interface.

In a further embodiment, the implantable neural interface further includes a voltage rectifier that co-optimizes the receiver antenna and the wireless data transmission to maximize power-transfer efficiency.

In a further embodiment, the several antennas include a dual-antenna architecture that minimizes the interference between the power link and the data TX.

In a further embodiment, the transceiver is placed on top of an analog recording and stimulation front-end (AFE) unit comprising the electrode array.

In a further embodiment, the receiver antenna is on-chip.

In a further embodiment, the transmitter antenna is on-chip.

In a further embodiment, the transceiver is implemented on a single CMOS silicon chip and components for power delivery, energy storage, data communication, including an antennas, are implemented on the same chip.

In a further embodiment, the receiver antenna is a loop antenna, and the transmitter antenna is a dipole antenna.

In a further embodiment, the receiver and transmitter antenna use different polarizations to maximize isolation.

In a further embodiment, the front end circuitry and the transceiver define a rectangular form factor comprising: a skull facing side with the transceiver; and a brain facing side with the front end circuitry and the electrode array.

In a further embodiment, the implantable neural interface further includes a housing, where: the several electrodes are associated with an exterior surface of the housing; and the front end circuitry and the transceiver are associated with an interior of the housing.

In a further embodiment, the electrode array, the front end circuitry, and the transceiver are embodied as a system on a chip (SOC).

In another embodiment, an implantable medical device includes: an electrode array having several electrodes; front end circuitry including: one or more digital components, and at least one amplifier coupled to a first electrode and a second electrode of the electrode array, where the amplifier and the first electrode and the second electrode form a sensing channel configured to sense electrical activity; and a transceiver including: several digital components; a power harvesting system that receives RF energy through an inductive wireless link; a wireless clock receiver that provides a clock signal to the one or more digital components of the front end circuitry and the several digital components of the transceiver; and a data transmitter configured to transmit data corresponding to the sensed electrical activity.

In a further embodiment, the electrode array is configured for implant in or on a heart, and the sensed electrical activity corresponds to electrical cardiac activity.

In a further embodiment, the electrode array is configured for implant in or on a brain, and the sensed electrical activity corresponds to electrical neural activity.

In a further embodiment, the electrode array is configured for implant in or on a spine, and the sensed electrical activity corresponds to electrical neural activity.

In yet another embodiment, a fully integrated system-on-chip (SOC) includes: a data transceiver (TRX) that is powered through a radio frequency (RF) power link, including: a power-harvesting system including a rectifier and a power management unit (PMU); a data receiver (RX) for receiving data from an external reader on a wireless downlink (DL); a reconfigurable data transmitter (TX) for transmitting data to the external reader on a wireless uplink (UL); several antennas that enable simultaneous power delivery and data communication through two distinct wireless links separated in the frequency domain; a receiver antenna that is shared between the power harvesting system and the RX; where the TX includes a power oscillator (PO) directly connected to a transmitter antenna for wireless data transmission.

In a further embodiment, the DL data is incorporated into the power link with an amplitude-shift-keying (ASK) modulation scheme.

In a further embodiment, simultaneous UL and DL communication is enabled using frequency division duplexing (FDD), where a center frequency of the UL is in the GHz region.

In a further embodiment, the TX is configured to transmit UL data with either on-off-keying (OOK) and ultrawideband (UWB) modulation.

In a further embodiment, the RX is directly powered by the power-harvesting system and is active during the entire operation of the system.

In a further embodiment, the SOC includes a voltage rectifier that co-optimizes the power receiving antenna and the wireless data transmission to maximize power-transfer efficiency.

In a further embodiment, the PMU converts unregulated output voltage of the rectifier to a constant dc voltage and adjusts the power consumption of the system.

In a further embodiment, the PMU sets the operating mode and biasing condition for different components of the TRX based on their power consumption and the total available power budget.

In a further embodiment, depending on the power consumption of each block, the PMU sets its power delivery scheme to either continuous or duty cycled.

In a further embodiment, the SOC further includes a storage capacitor (Cₛ) that stores converted energy by the rectifier and a voltage limiter is included in the PMU to prevent voltage breakdown, where the PMU monitors the voltage level across Cₛ and establishes active and sleep modes for the TX operation.

In a further embodiment, the several antennas include a dual-antenna architecture that minimizes the interference between the power link and the TX.

In a further embodiment, the TRX uses an amplitude-based modulation scheme to maximize energy efficiency.

In a further embodiment, the TRX is placed on top of an analog recording and stimulation front-end (AFE) unit with a 2D microelectrode array comprising a plurality of electrodes.

In a further embodiment, the TRX acts as a communication hub between electrodes within an AFE unit and the external reader.

In a further embodiment, the receiver antenna is on-chip.

In a further embodiment, the transmitter antenna is on-chip.

In a further embodiment, the TRX is implemented on a single CMOS silicon chip and components for power delivery, energy storage, data communication, including an antennas, are implemented on the same chip.

In a further embodiment, the receiver antenna is a loop, and the transmitter antenna is a dipole.

In a further embodiment the receiver and transmitter antenna use different polarizations to maximize isolation

In yet another embodiment, a fully integrated system-on-chip (Soc) for neural stimulation and recording, includes: a power harvesting system that receives RF energy through an inductive wireless link; a wireless clock receiver that provides a clock signal to a plurality of digital components; an amplifier that senses neural activity and increases a voltage amplitude of a signal;

In a further embodiment, the system-on-chip further includes an Analog to Digital Converter (ADC) that converts an amplified signal to a digital signal.

In a further embodiment, the ADC is a Successive Approximation Register (SAR) ADC.

In a further embodiment, the system-on-chip further includes a Parallel-Input-Serial-Output (PISO) shift register unit that loads digitized data from the recording channels and passes the serialized data stream to the data TX.

In a further embodiment, the data TX is an ultra-wideband (UWB) transmitter operating in the 3-10 GHz range.

In a further embodiment, the data TX operates in the ISM bands between 10 MHz and 10 GHz.

In a further embodiment, the data TX uses at least modulation scheme selected from the group consisting of on-off-keying (OOK), amplitude-shift-keying (ASK), and UWB modulation.

In a further embodiment, an operating mode of the transmitter is controlled by an external mode selection signal.

In a further embodiment, control commands provide information on the start and end time of the neural sensing, clock rate, number of bits for digitization, the frequency of the transmitter, and the modulation of the transmitter signal.

In a further embodiment, the controlled commands are generated by an external device and transmitted wirelessly to the SOC.

In a further embodiment, the voltage waveforms of heart are detected and amplified.

In a further embodiment, the voltage waveforms of an implantable sensor is measured and amplified.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1A illustrates a closed-loop neural recording and stimulation system based on a distributed neural interface in accordance with an embodiment of the invention.

FIG. 1B illustrates a system for heart signal detection in accordance with an embodiment of the invention.

FIB. 1C illustrates a system that includes an electrode array configured for implant in or on a heart, and sensed electrical activity corresponds to electrical cardiac activity in accordance with an embodiment of the invention.

FIG. 2 illustrates a system architecture of a wireless neural recording system-on-chip (SoC) in accordance with an embodiment of the invention.

FIG. 3A illustrates a circuit schematic of a power harvesting system alongside a clock recovery system in accordance with an embodiment of the invention.

FIG. 3B illustrates a circuit implementation of a clock recovery system and internal waveforms in accordance with an embodiment of the invention.

FIG. 4A illustrates various electrode placement for neural signal acquisition in accordance with an embodiment of the invention.

FIG. 4B illustrates their corresponding frequency content and voltage amplitude in accordance with an embodiment of the invention.

FIG. 5 illustrates a circuit schematic of a single channel analog data front-end in accordance with an embodiment of the invention.

FIG. 6 illustrates a circuit schematic of a UWB data transmitter alongside with the internal nodes waveforms in accordance with an embodiment of the invention.

FIG. 7 illustrates a closed-loop neural recording and stimulation system for neural prostheses in accordance with an embodiment of the invention.

FIG. 8 illustrates a circuit architecture of a wirelessly powered frequency division duplexing (FDD) radio in accordance with an embodiment of the invention.

FIG. 9 illustrates a timing diagram of PMU internal nodes in different power deliver schemes, including (A) duty cycled and (B) continuous in accordance with an embodiment of the invention.

FIG. 10 illustrates a process for optimization of a power-transfer link in accordance with an embodiment of the invention.

FIG. 11 illustrates a design of an on-chip coil and an on-chip dipole antenna in accordance with an embodiment of the invention.

FIG. 12 illustrates a simulation configuration of downlink (DL) and uplink (UL) wireless links in IE3D along with layer information of the simulation setup and a detailed view of a chip model in accordance with an embodiment of the invention.

FIG. 13 illustrates maximum power-transfer efficiency of a DL path for various tissue types in accordance with an embodiment of the invention.

FIG. 14 illustrates maximum efficiency of a UL patent for various tissue types in accordance with an embodiment of the invention.

FIG. 15 illustrates a circuit architecture of a power-harvesting system in accordance with an embodiment of the invention.

FIG. 16 illustrates a circuit architecture and timing diagram of an amplitude-shift-keying (ASK) data receiver (RX) block in accordance with an embodiment of the invention.

FIG. 17 illustrates switching status of a passive mixer in (A) positive and (B) negative cycles of an input RF signal in accordance with an embodiment of the invention.

FIG. 18 illustrates a circuit architecture of a Schmitt Trigger in the RX in accordance with an embodiment of the invention.

FIG. 19 illustrates a circuit architecture of a power oscillator (PO) alongside an equivalent circuit model in accordance with an embodiment of the invention.

FIG. 20 illustrates a circuit architecture of transmitter (TX) blocks and internal waveforms in both modulation schemes including (A) on-off keying (OOK) and (B) ultrawideband (UWB) in accordance with an embodiment of the invention.

### DETAILED DESCRIPTION OF THE DRAWINGS

Turning now to the drawings, closed-loop neural recording and stimulation systems in accordance with various embodiments of the invention are illustrated. Emerging applications of Brain Machine Interface (BMI) systems show an ever-increasing demand for neural activity acquisition with higher data-rates and better spatio-temporal resolutions. Accordingly, many embodiments provide a fully integrated, wireless, RF-powered data transceiver for high-performance implants, such as neural interfaces. In many embodiments, the design can occupy a total volume of 1.6 mm3 without a need for an off-chip component. In many embodiments, the integrated circuit (IC) receives power and downlink data with amplitude-shift-keying (ASK) modulation by an on-chip coil (OCC) through an RF wireless link. In many embodiments of the system, for uplink data transmission, the system can use a transmitter (TX) that can be designed based on a power oscillator stage directly connected to an on-chip dipole antenna that supports various data rates with both on-off-keying (OOK) and ultrawideband (UWB) schemes. The radio can include a power receiver (RX) system that enables the IC to operate under various power budgets by adjusting the duty cycle of the TX. For example, with a 25-dBm power TX at a 1-cm distance, the RX can achieve a maximum data rate of 2.5 Mbps with a power consumption of 2.6 μW. Also, in many embodiments, the TX can support data rates of up to 150 Mbps with UWB modulation with a 15-cm operating range achieving an energy efficiency of 4.7 pJ/b. Many embodiments of the system can improve RX and TX energy inefficiencies by ×50 and ×2.3, respectively.

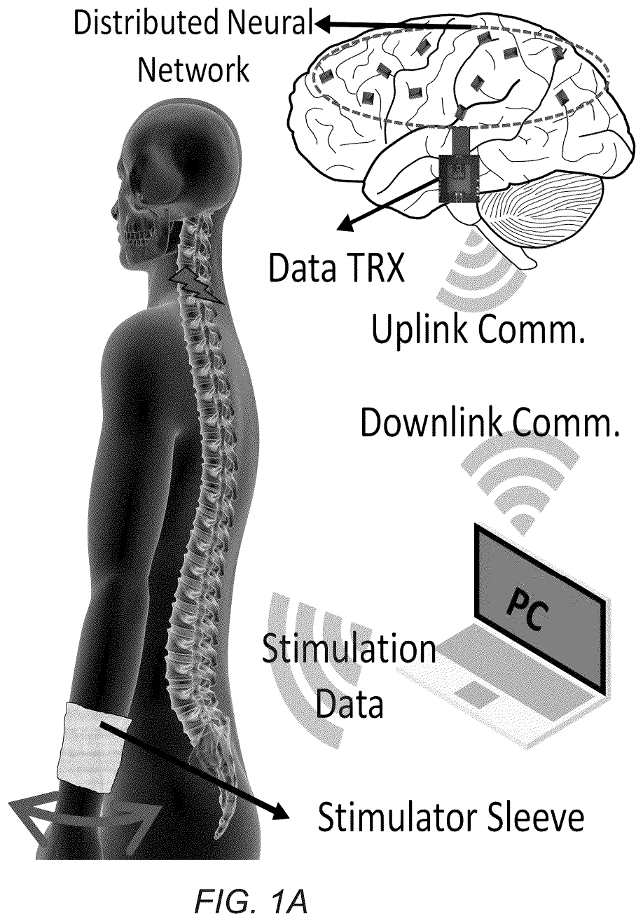

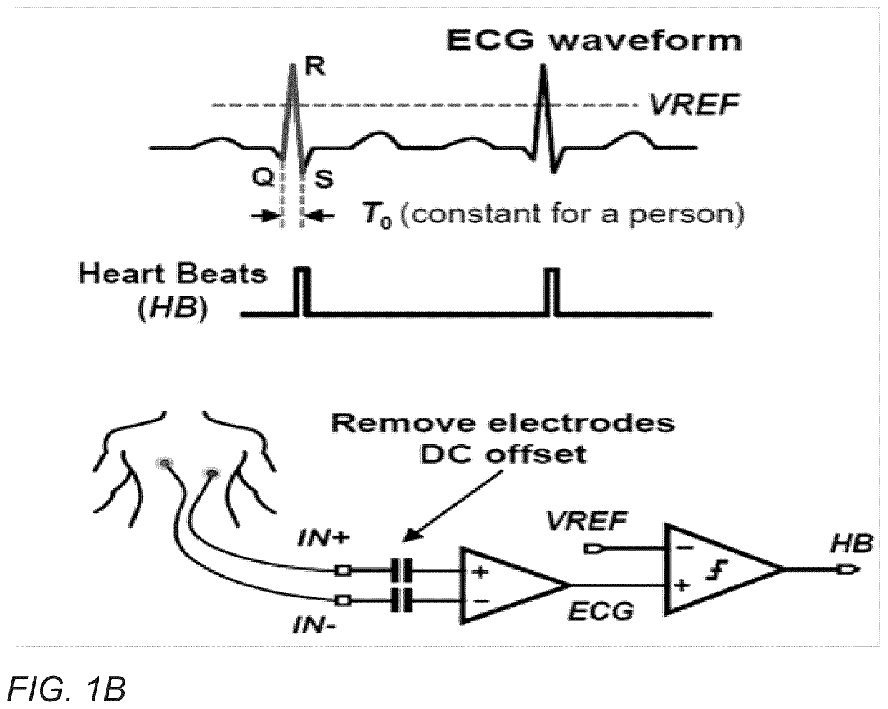

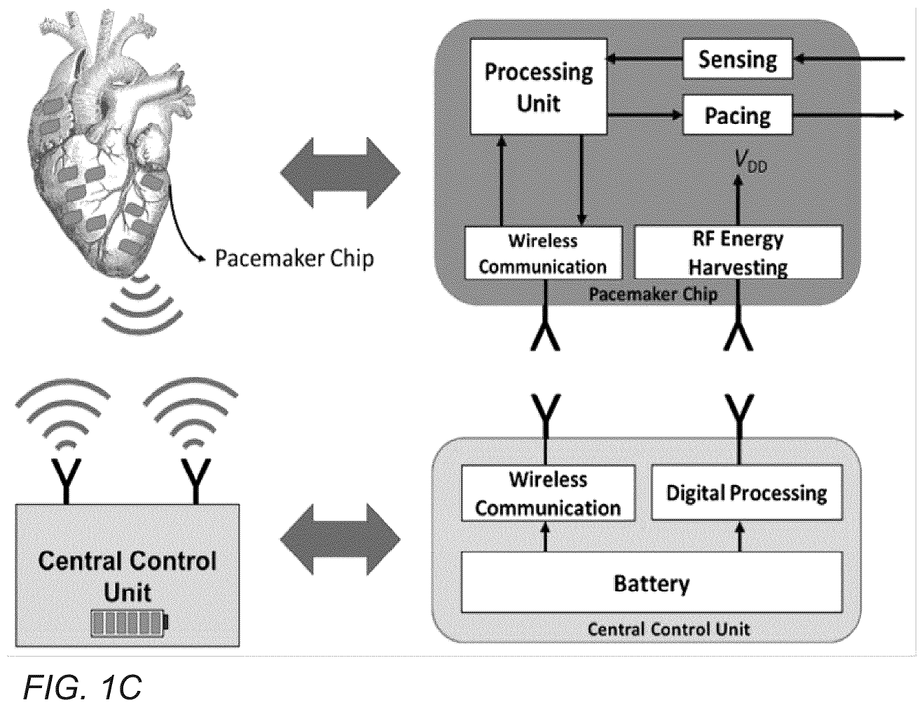

A closed-loop neural recording and stimulation system for patients who suffer from spinal cord injuries in accordance with an embodiment of the invention is illustrated in FIG. 1A. In many embodiments, brain signals can be recorded by a distributed neural recording system and the data may be transmitted to an external processor wirelessly. After the brain signals are decoded, arm muscles can be stimulated by an electronic sleeve wrapped around a patient's arm in order to help the patient regain control over their hand movement. Providing a real-time hand control relies mainly on acquiring neural activity with a high throughput at a fine scale. In a distributed sensor network scheme, miniaturizing the individual recording nodes can be an effective solution that renders more recording sites and improves the resolution. Accordingly, many embodiments provide for a distributed neural recording system based on a fully integrated system-on-chip (SoC).

In many embodiments, the system can be used to record and stimulate cardiac activity. FIG. 1B illustrates a circuit architecture for heart signal detection in accordance with an embodiment of the invention. A closed-loop recording system that includes an electrode array that is configured for implant in or on a heart, and that senses electrical activity that corresponds to electrical cardiac activity in accordance with an embodiment of the invention is illustrated in FIG. 1C. As illustrated the system includes a central control unit that communicates with a pacemaker chip that can be implanted on or in a heart. The pacemaker chip can include a processing component, a sensing component, a pacing component, a wireless communication component, and an RF energy harvesting component. The central control unit can include a wireless communication component, a digital processing component, and a battery. Although FIG. 1C illustrates a particular recording system architecture for an implant on a heart, any of a variety of system architectures can be utilized as appropriate to the requirements of a specific application in accordance with various embodiments of the invention.

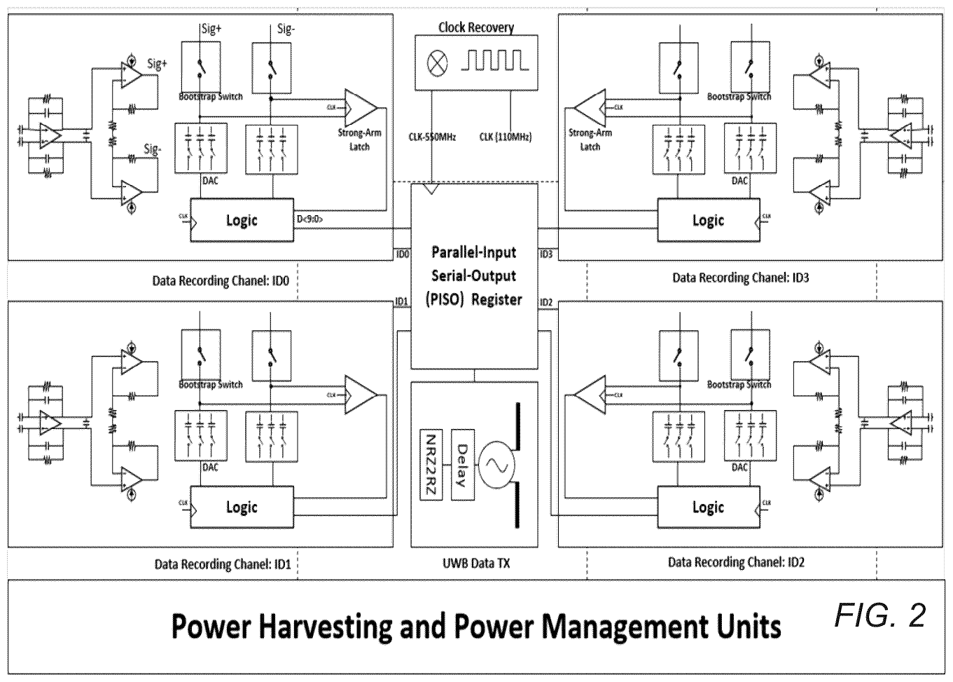

A block diagram of a fully integrated SoC in accordance with an embodiment of the invention is illustrated in FIG. 2. In many embodiments, the SoC can be fabricated on a commercial CMOS technology and can be composed of the following components: 1) a power harvesting system that can receive RF energy through an inductive wireless link; 2) a wireless clock receiver that can provide an accurate clock signal for the digital components of the SoC with no external components such as a crystal oscillator; 3) a four-channel low-power and low-noise-amplifier (LNA) that can sense neural activity and increase the voltage amplitude; 4) a four-channel Successive Approximation Register (SAR) Analog to Digital Converter (ADC) that can convert the amplified signal by the LNAs to an N bit (e.g., 10-bit) digital signal; 5) a Parallel-Input-Serial-Output (PISO) shift register unit that can load the digitized data from several (e.g., four) recording channels and pass the serialized data stream to a data transmitter; and 6) an Ultra-Wideband (UWB) data transmitter for wireless transmission of the recorded data to an external receiver. Although FIG. 2 illustrates a particular circuit architecture of an integrated SoC with certain components, any of a variety of circuit architectures can be specified as appropriate to the requirements of a specific application in accordance with various embodiments of the invention.

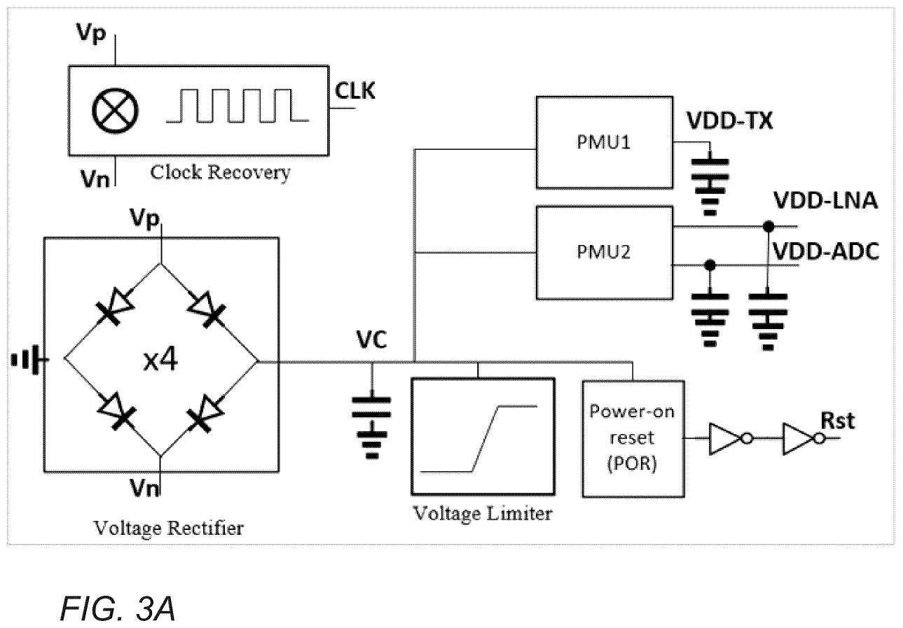

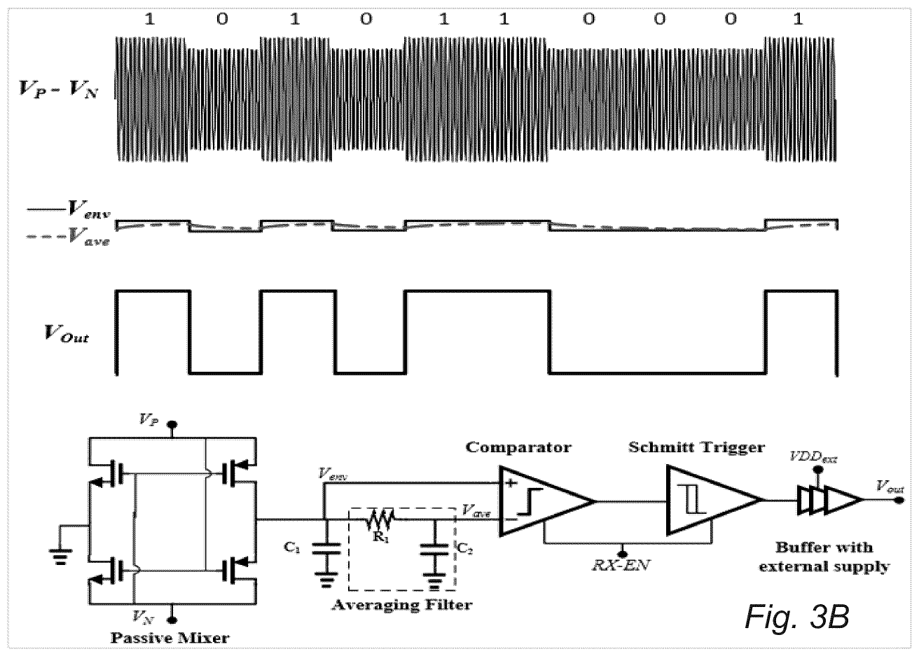

To realize an SoC with minimal cost and a low footprint, wireless power and clock signal for the incorporated digital circuitry can be transferred to the SoC through an inductive wireless link. In many embodiments, an SoC may be equipped with a power harvesting system and a clock recovery circuitry. A circuit schematic of a power harvesting system along with the clock recovery system in accordance with an embodiment of the invention is illustrated in FIG. 3A. The wireless link can be modulated with a 550 KHz square wave with a modulation index of less than 20%. Once the RF signal is received by a power receiving coil, it can be passed to a voltage rectifier that converts the RF energy to a dc signal. However, in many embodiments, the rectifier cannot be directly used for powering sensitive circuits in a neural recording SoC since the voltage may be easily varied with load variation. Hence, in many embodiments, low dropout voltage regulators can be utilized to ensure the dc voltage level is stable. On the other hand, to ensure the supply voltage of data front-end remains constant during data transmission, many embodiments may incorporate a dual-LDO architecture. The general building blocks of an SoC such as LNAs, shift register, ADCs and the clock recovery can be powered by a power management unit (PMU) that delivers different voltage levels, for example two voltage levels of 1.1V and 1V. In many embodiments, the TX block can be powered by a different LDO that generates a different voltage, for example a 1.3 V supply voltage. Since the LDOs can be decoupled from each other, the fast load transition caused by data TX may not show any effect on the supply voltage of the general blocks. In many embodiments, the power receiving antenna can be shared between the power harvesting system and the clock recovery circuit. Hence, the clock recovery circuit may extract the envelope of the RF signal and convert it to a rail-to-rail square wave that can be used as an accurate clock signal. The operation of the clock recovery circuit in accordance with an embodiment of the invention is illustrated in FIG. 3B. The recovered 550 KHz clock signal can also be divided by five to generate a 110 KHz square wave for the operation of individual ADCs that have a sampling rate of 10 KSps. Although FIG. 3A illustrates a particular circuit schematic of a power harvesting system along with the clock recovery system, any of a variety of circuit architectures may be utilized as appropriate to the requirements of a specific application in accordance with various embodiments of the invention.

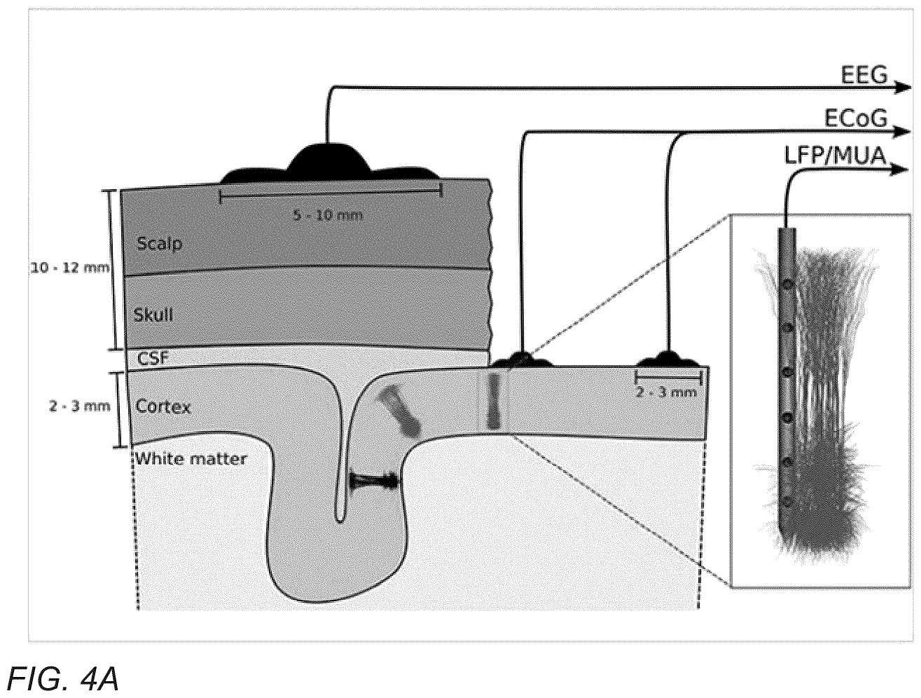

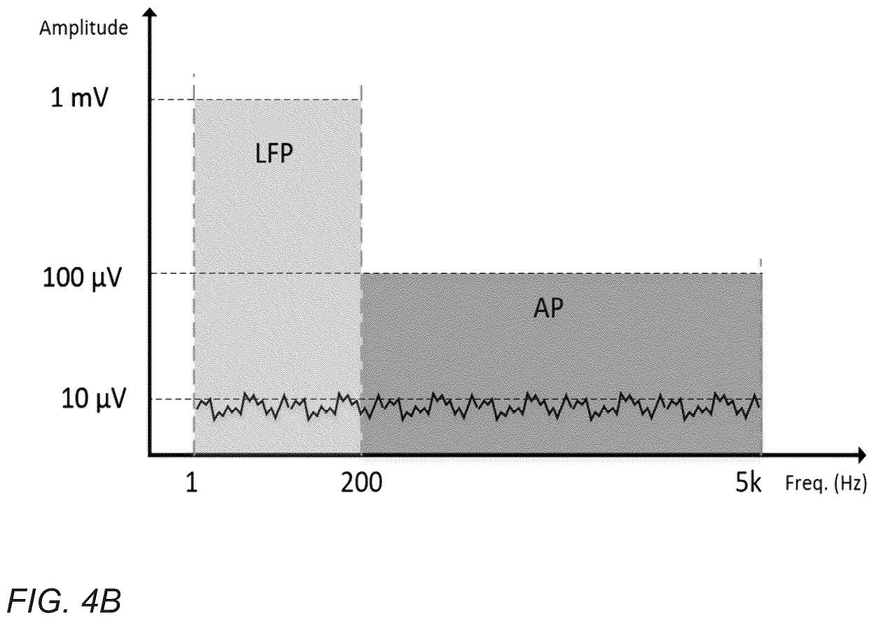

FIG. 4A illustrates various electrode placements for neural signal acquisition and FIG. 4B illustrates their corresponding frequency content and voltage amplitude in accordance with an embodiment of the invention.

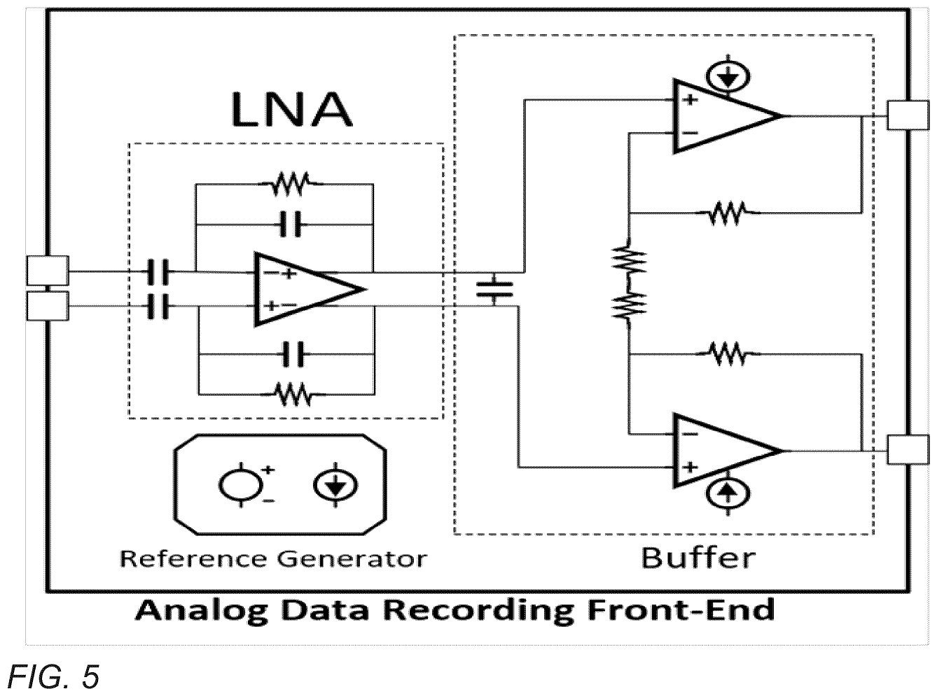

Neural signals may contain energy at different frequencies and depending on the recording type and the placement of the electrodes they induce various voltage levels at the recording site. In many embodiments, the low-amplitude sensed signals may need to be amplified before digitization by an ADC. Since the full-scale voltage of the ADC in accordance with many embodiments may be set to 1V, the amplifier should provide at least 60 dB of gain. On the other hand, the input referred noise of such an amplifier should remain below the background noise of the human brain which is about 10 μVrms. Although the amplifier should be able to boost the signal frequency content higher than 1 Hz, the DC component of the electrodes signal should be removed. Hence, many embodiments utilize pseudo-resistors to realize a Giga-ohm level resistance on a silicon chip and achieve a low corner frequency for the amplifier. The analog data front-end in accordance with many embodiments of the invention can be implemented based on a fully differential scheme. A circuit schematic of an analog data front-end with a fully differential scheme in accordance with an embodiment of the invention is illustrated in FIG. 5. In many embodiments, the total power consumption of the LNA is 1 μW and it provides a 40 dB gain. To increase the driving capability of the analog data front-end (DFE), the ADC may be followed by a buffer circuitry with the power consumption of 1 μW that provides an additional gain of 20 dB and enables the DFE to drive the capacitive load of a SAR ADC. Although FIG. 5 illustrates a particular circuit architecture of an analog data front-end implemented based on a fully differential scheme, any of a variety of circuit architectures may be utilized as appropriate to the requirements of a specific application in accordance with various embodiments of the invention.

In many embodiments, the data amplified signal can be digitized with a 10-bit SAR ADC that is realized with an extremely low power consumption of 290 nW. The ADC may achieve an Effective Number of Bits (ENoB) of 9.3 Bits with a Signal-to-Noise-and-Distortion-Ratio (SNDR) of 57.7 dB.

In many embodiments, the digitized data by the four ADCs can be passed to a PISO shift register and may be combined with channel identifiers and preamble data. As a result, between two consecutive conversions of an ADC, the shift register may pass 55 bits to the UWB transmitter to be wirelessly transferred.

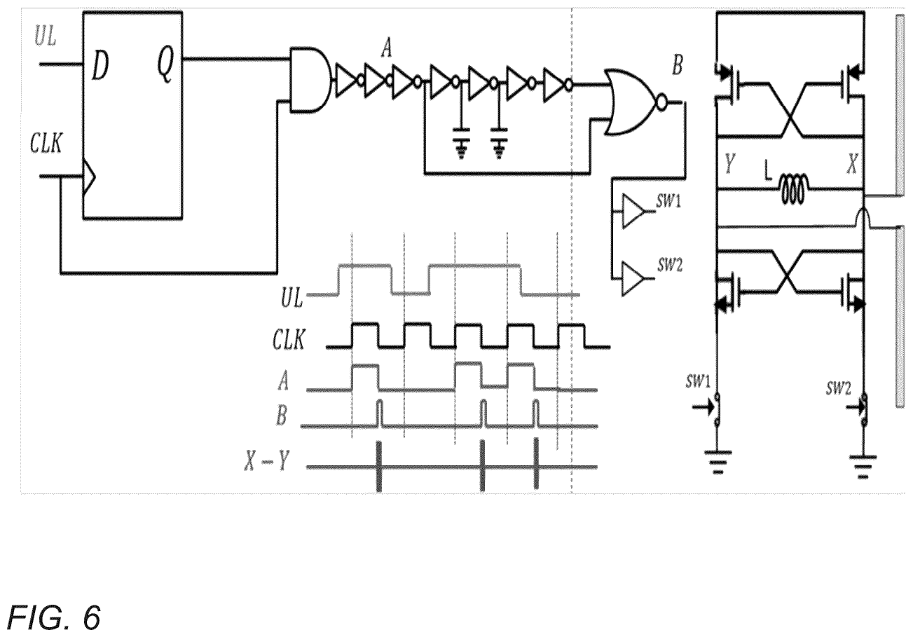

A circuit schematic of an UWB TX and its timing diagram in accordance with an embodiment of the invention are illustrated in FIG. 6. To save energy and enhance the energy efficiency of the data telemetry, the TX block may be equipped with a Non-Return-to-Zero (NRZ) to Return-to-Zero (RZ) converter to introduce a rising edge for consecutive bits of “1”. The NRZ to RZ convertor can be followed by a pulse generator that generates a 50 ns short pulse upon the detection of a rising edge. The generated pulses may be utilized to activate a power oscillator circuitry that drives an on-chip dipole antenna. Hence, in many embodiments, for transmission of a “1” bit, the power oscillator only remains active for a very short period that results in an extensive average power reduction. Although FIG. 6 illustrates a particular circuit schematic of an UWB TX, any of a variety of circuit architectures may be utilized as appropriate to the requirements of specific applications in accordance with various embodiments of the invention. various applications in accordance with various embodiments of the invention.

In many embodiments, the SoC can be fabricated in a 180-nm CMOS technology. Considering the mm-sized form-factor of the SoC and its ability of wireless operation, the SoC in accordance with many embodiments can be utilized for low-power and miniaturized neural recording systems.

Wirelessly Powered Reconfigurable Frequency Division Duplexing (FDD) Radio with On-Chip Antennas for Multi-Site Neural Interfaces

As noted above, wireless operation is a necessity for human implantable systems. Therefore, commercial implants can utilize either batteries or inductive coupling through a pair of coils for powering the internal electronics. Also, bidirectional data transmission can be conducted through a wireless link that utilizes electromagnetic (EM) antennas. Advances in semiconductor technology have resulted in a significant integration capability and size reduction of electronics. However, the overall size of an implant may not be scaled with the same rate since it is dominantly controlled by the size of the required components for powering and data communication. Battery-powered devices may not be made smaller than a few centimeters since the power density of state-of-the-art batteries fails to address the demands of long-term miniaturized implants. On the other hand, the efficiency of power-transfer systems is proportional to the dimension of power receiver (RX) and transmitter (TX) structures. Compared with traditional inductively coupled wireless power-transfer (WPT) systems that utilize cm-sized structures, high-frequency WPT systems can incorporate mm-sized antennas at the cost of a lower efficiency.

A requirement of neural implants is improving the spatiotemporal resolution of the recorded signals to provide more insight into the complex mechanism of human functions. A higher spatiotemporal resolution necessitates signal recording from a smaller area with a higher recording rate. To enable recording at a fine scale, many embodiments of the invention provide implantable system-on-chips (SoCs) to realize a distributed neural recording system. The system-level requirements of such SoCs in accordance with many embodiments are long-term wireless operation, mm-sized form factor, and integration capability on a commercial CMOS process to make them scalable and cost-efficient. Miniaturizing the size of an implant can address the needs of implantable systems since it can result in a higher sensor density and also enables signal recording at an ultra-small structural scale.

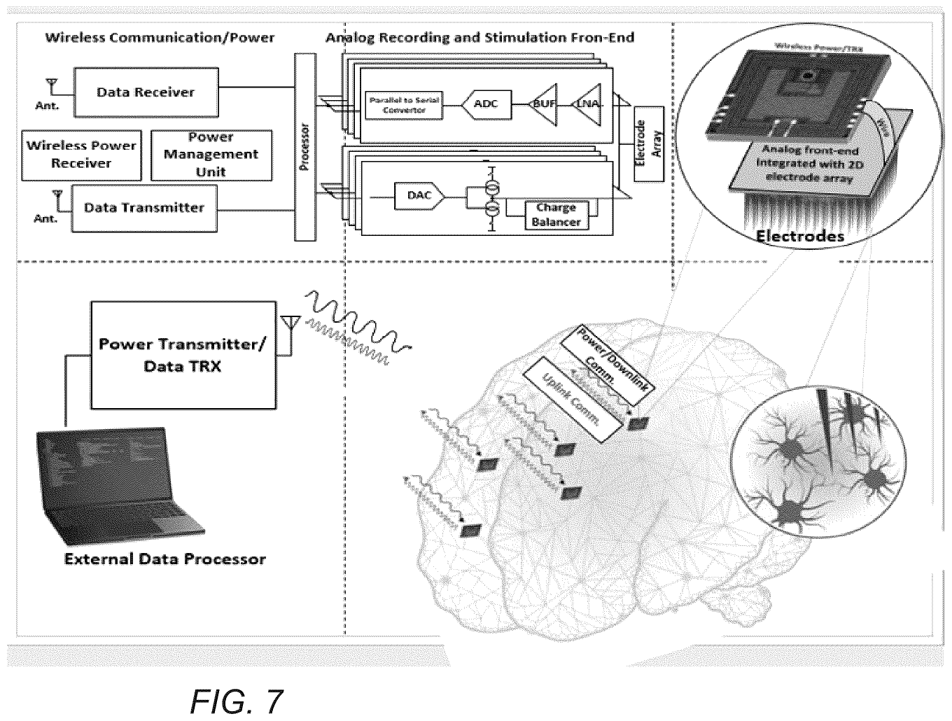

FIG. 7 illustrates a conceptual multi-site and distributed neural interface enabled by multiple mm-sized recording/stimulating units in accordance with an embodiment of the invention. Each unit can include a data transceiver (TRX) that is placed on top of a microelectrode array. Because of the compact size, individual units can be tightly placed on the brain to improve the recording resolution. There are various challenges toward realizing a practical neural interface system. Accordingly, many embodiments of the system address the data communication problem and provide a TRX compatible with the system-level requirements of implantable systems. In many embodiments, each TRX can acts as a communication hub between the electrodes within a unit and an external reader. Recent neural recording systems have reported up to 4096 recording electrodes. On the other hand, various recording methods such as ECoG or spike recording demand different sampling rates that may reach as high as 10 ksps per channel. Hence, the overall communication bandwidth may exceed tens of Mbps.

A practical TRX in accordance with many embodiments of the invention should support the demanded bandwidth and be compatible with most all of the system-level requirements of miniaturized implants. Achieving such a high data rate can be difficult due to severe power budget constraints and poor performance of electrically small antennas used for power-harvesting and data communication. Considering the specific absorption-rate (SAR) limit of various biological tissues and nonidealities of a WPT system such as coil misalignment and link variations, the maximum harvested power by mm-sized power harvesters can be about few hundreds of micro-Watts. Moreover, the wavelength of EM waves at the frequencies that data communication is typically conducted ranges from tens to hundreds of centimeters. A mm-sized antenna is often much smaller than the wave-length and has poor radiation efficiency. Accordingly, many embodiments provide for a TRX for mm-sized implants that can achieve a very high-energy efficiency to support high data rates.

Backscattering is a widely adopted technique for telemetry in implantable applications since it results in extremely low-power consumption. The transmitted data pattern can be used for load shift keying (LSK) modulation of the power coil and alters the reflected signal to an external reader. Despite achieving a superior energy efficiency over active communication, backscattering radios fail to address the main requirements of mm-sized implants. Due to the small size of the power coil and a strong power carrier, which acts as a blocker, detection of the reflected signal on the reader side may be difficult or even impossible. In addition, modulating the power coil disrupts the power flow into the system and degrade power-transfer efficiency. Furthermore, the communication bandwidth of backscattering radios is often very low due to the high-quality factor (Q) of the power coil that limits the data rate, consequently.

Active TRXs may not face the challenges of their backscattering counterparts and can potentially achieve high data rates at the expense of higher power consumptions. Considering the stringent power budget in implantable applications, a design goal of many embodiments of the system can be achieving the highest possible energy efficiency; hence, proper modulation schemes can be chosen. There is a trade-off between energy efficiency and spectral efficiency in communication systems. Narrowband modulation schemes demand a relatively complex architecture to generate an accurate frequency whereas wideband modulation schemes such as on-off keying (OOK) have often less complexity and result in higher energy efficiency.

Accordingly, many embodiments provide for the design, implementation, and verification of a fully integrated and RF-powered wireless data TRX. The radio in accordance with many embodiments of the system can achieve state-of-the-art energy efficiency and the smallest form factor compared with prior mm-sized wirelessly powered active radios.

In many embodiments, the system can be implemented on a single CMOS silicon chip and all needed components for power delivery, energy storage, and data communication, including an on-chip coil (OCC) and a dipole antenna, can be implemented on the same chip. The TRX in accordance with many embodiments of the system can be designed to enable simultaneous power delivery and data communication through two distinct wireless links separated in the frequency domain. In many embodiments, the system design can support data rates of up to 2.5 Mbps in the RX and data rates of up to 150 Mbps in the TX chain, respectively. In many embodiments, the system can occupy a total area of 2.4 2.2 0.3 mm³ without any substrate thinning and features a fully on-chip integration that can result in cost reduction, elimination of any post-fabrication process, and reliability improvement among other benefits.

Described below is an overview of the TRX in accordance with many embodiments and the high-level system operations. Also described are the wireless link design and frequency selection considerations for power delivery and data communication. Also described are design details of incorporated circuitry in the power harvesting system, RX, and TX.

### System-Level Description

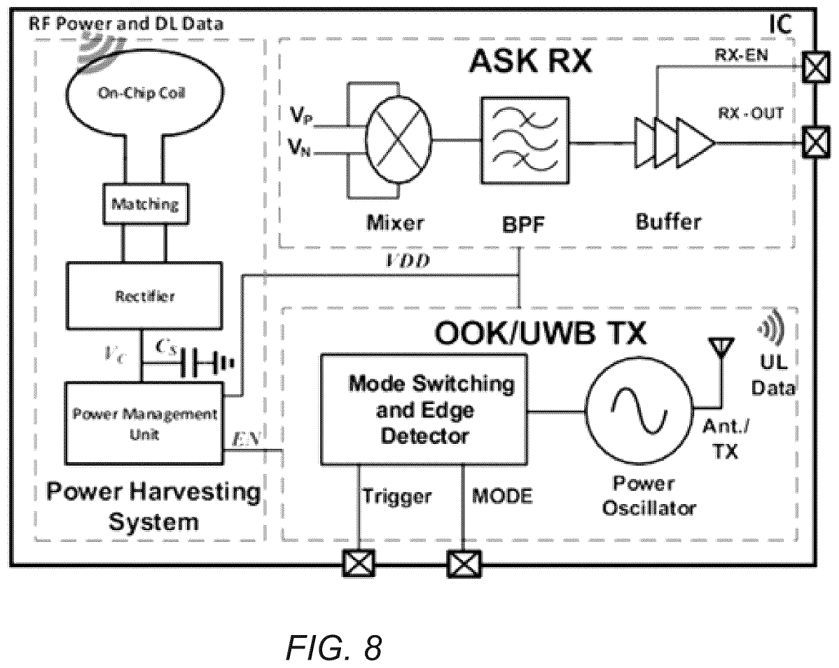

A high-level block diagram of a wirelessly powered TRX in accordance with an embodiment of the invention is illustrated in FIG. 8. In many embodiments, the TRX is powered through a radio frequency (RF) link. The system can incorporate a power-harvesting system, composed of a rectifier and an on-chip antenna (rectenna) and a power management unit (PMU), a data RX, and a reconfigurable data TX. Besides, two antennas can be integrated on the same chip to enable simultaneous power delivery and data communication. An OCC can be shared between the power-harvesting system and the RX while the TX utilizes a dipole antenna for wireless transmission. The TRX in accordance with many embodiments of the invention can be used for medical applications, and features a fully integrated design with a mm-sized form factor. Considering the losses of biological tissues at high frequencies and the scarcity of the harvested power by a mm-sized power-harvesting system, achieving a high-throughput wirelessly powered TRX may benefit from a comprehensive design strategy. Accordingly, many embodiments, rather than focusing on the individual circuit blocks, have adopted a joint design approach for the wireless link and internal circuit blocks. Described below include how the mm-sized constraints can dictate the operating frequency and many design specs of the TRX in accordance with many embodiments of the system. In the system design in accordance with several embodiments of the invention, a goal is to achieve energy-efficient data communication in both RX and TX chains to enable high-throughput wireless communication under severely restricted power budgets rendered by a mm-sized power-harvesting system. Data modulation schemes and TRX architectures in accordance with many embodiments of the system can be carefully chosen to minimize circuit complexity and overall power consumption.

In many embodiments, a robust operation can be enabled by the following techniques:
(1) co-optimizing the power receiving coil and the wireless link with voltage rectifier to maximize power-transfer efficiency; (2) employing a PMU to set the operating mode and biasing condition of different blocks depending on their power consumption and the total available power budget; (3) utilizing a dual-antenna architecture to minimize the interference between the power link and the TX; (4) exploiting amplitude-based modulation schemes in the TRX for maximizing energy efficiency; (5) utilizing an architecture based on a power oscillator (PO) in the TX block to achieve the highest possible energy efficiency; and 6) applying circuit-level power reduction techniques in the PO design.

The needed data rate of TX and RX paths in medical implants can vary considerably and thus the communication can be asymmetric. In many embodiments, the wireless link from an external reader to the RX, hereinafter referred to as downlink (DL), can demand a data rate that does not exceed a few Mbps. On the other hand, the wireless link from the TX to an external reader, hereinafter referred to as uplink (UL), may require a large bandwidth to support data rates up to hundreds of Mbps. In many embodiments, the DL data can be incorporated into the power link with an amplitude-shift-keying (ASK) modulation scheme. The RX block can be directly powered by the power-harvesting system and can be active during the entire operation of the system. Hence, it can be imperative to minimize the overall power consumption of the RX. To enable simultaneous UL and DL communication, many embodiments of the system can exploit the frequency division duplexing (FDD) technique and set the center frequency of the UL in the GHz region. Such a high center frequency may alleviate the undesired effects of the strong power link on the TX communication and minimize the interference of UL and DL. Besides, the efficiency of a mm-sized antenna can improve as the frequency increases to the GHz region. In many embodiments of the system, the UL communication can incorporate amplitude-based modulation schemes due to their superior energy efficiency and less sensitivity to supply variation as opposed to frequency-based modulation schemes. In many embodiments, the TX block can be configured to transmit UL data with either OOK or ultrawideband (UWB) modulation, among various other modulation techniques.

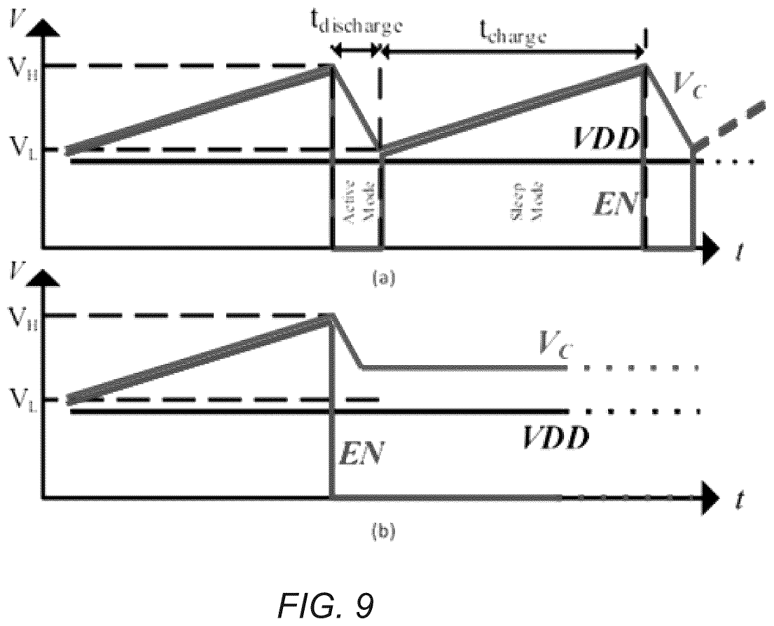

In many embodiments, the PMU can convert the unregulated output voltage of the rectifier to a constant dc voltage and can adjust the power consumption of the entire system. The maximum harvested power in mm-sized implants is often less than the power consumption of a power-hungry block such as a data TX. Many embodiments can address this problem by duty cycling the operation of power-demanding blocks and lowering the overall power consumption of the system. Depending on the power consumption of each block, the PMU can set its power delivery scheme to either continuous or duty cycled. In many embodiments, a storage capacitor (C_{S}) can be used for storing the converted energy by the rectifier and a voltage limiter can be included in the PMU to prevent any voltage breakdown. In many embodiments of the system, the most power-demanding block of the system can be the data TX. Therefore, in many embodiments, the PMU can monitor the voltage level across C_{S}, and establish active and sleep modes for the TX operation. The waveforms of the internal nodes of the PMU in a duty cycled power delivery scheme in accordance with an embodiment of the invention are illustrated in FIG. 9 item (a). If the harvested power falls below TX power consumption, the TX block is periodically deactivated by the enable (EN) signal to allow the PMU to maintain V_{C} higher than a minimum threshold amount (V_{L}) that is required for continuous operation of the RX block and internal circuitry of the PMU. For the entire duration of the sleep mode (t_{charging}), the rectifier charges the C_{S} and V_{C} rises until it reaches a predefined threshold (V_{H}). If the harvested power is sufficient for continuous operation, EN stays low and V_{C} settles at a voltage level between V_{H} and V_{L} as illustrate in FIG. 9 item (b). Although FIG. 8 illustrates a particular architecture of a wirelessly powered FDD radio, any of a variety of architectures can be utilized as appropriate to the requirements of a specific application in accordance with various embodiments of the invention.

### Wireless Link Implementation

A wireless link in accordance with many embodiments of the invention can include two distinct antennas that are used in DL and UL paths. Prior mm-sized RF WPT systems featuring an OCC as power RX have reported an operating frequency in the order of few hundreds of MHz. To minimize the interference of the WPT system, many embodiments of the system can extend the operating frequency of the data TX to the GHz frequency region. Among various types of antennas that can be utilized in accordance with embodiments of the system, a dipole structure can be an attractive choice for the UL path due to its simple profile and complicity with on-chip integration. The dipole antenna can also be easy for on-chip implementation and has a small footprint. To enhance the harvested power for the system operation and maximize the data rate in the UL path, it can be imperative to optimize the antenna dimensions and operating frequency.

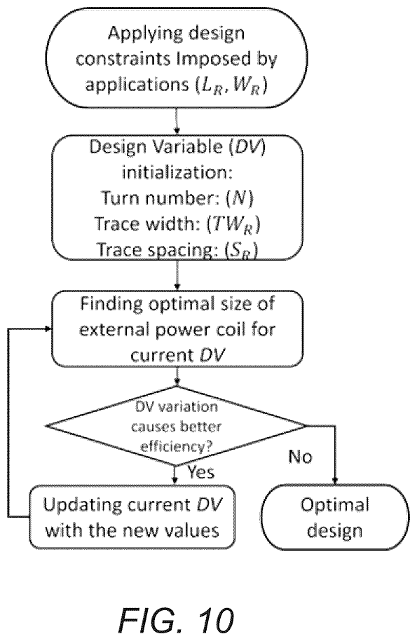

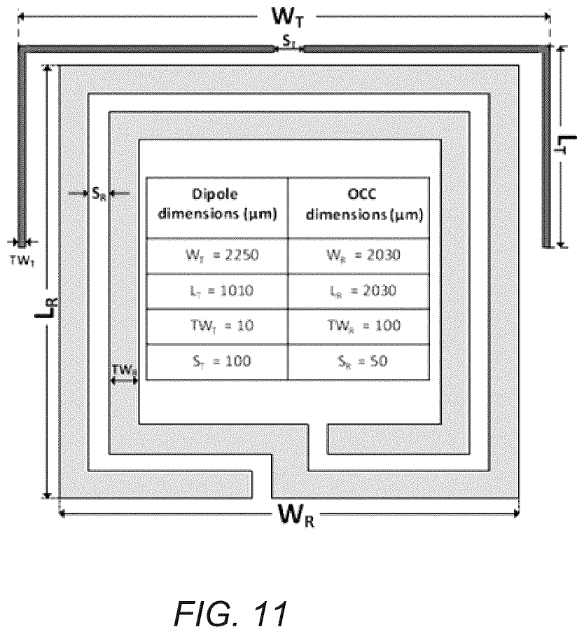

In prior systems, the wireless link is often modeled as a two-port network and the link optimization is conducted through an iterative algorithm that aims to maximize the power-transfer efficiency. The two-port network model fora wireless link is a general approach and can be applied to any wireless link regardless of near-field or far-field EM region operation and link composition surrounding the antennas. Accordingly, many embodiments of the system can use a two-port network model for both DL and UL design, and adopt an optimization algorithm. A flowchart of an optimization algorithm for the power link in accordance with an embodiment of the invention is illustrated in FIG. 10. Considering the mm-sized requirement, the maximum dimension can be limited to 2.25 mm and the distance between the external power TX and the OCC is set to 12 mm. Due to the relatively large coupling between the external coil and the OCC, the design variables of the OCC can be jointly optimized with the external power coil through an iterative optimization algorithm. For UL communication, many embodiments of the system can use an on-chip dipole that transmits TX data to an external UWB monopole antenna with a bandwidth of 3-7 GHz. The power-transfer efficiency of the WPT system can be susceptible to degradation by the presence of conductive material in the proximity of the power TX coil. To ensure the wireless power flow to the system is not altered by the UWB monopole antenna, many embodiments of the system can choose the UL communication distance as 15 cm. The optimized design for the dipole antenna can be achieved using a similar optimization algorithm as the WPT system. However, due to the large distance and a weak coupling between the dipole and monopole antennas, the design variables of the monopole antennas are not changed through the optimization process. An optimal dimensions of the OCC and the dipole antenna in accordance with an embodiment of the invention are illustrated in FIG. 11. Simulation results have shown the implemented OCC has an inductance value of 13.6 nH and achieves an unloaded Q-factor of 14.3 at 250 MHz. Although FIG. 11 illustrates a particular design of the on-chip coil (OCC) and an on-chip dipole antenna, any of a variety of design specification and types of antenna can be utilized as appropriate to the requirements of a specific application in accordance with various embodiments of the invention.

### Tissue Effect on Optimal Frequency and Efficiency

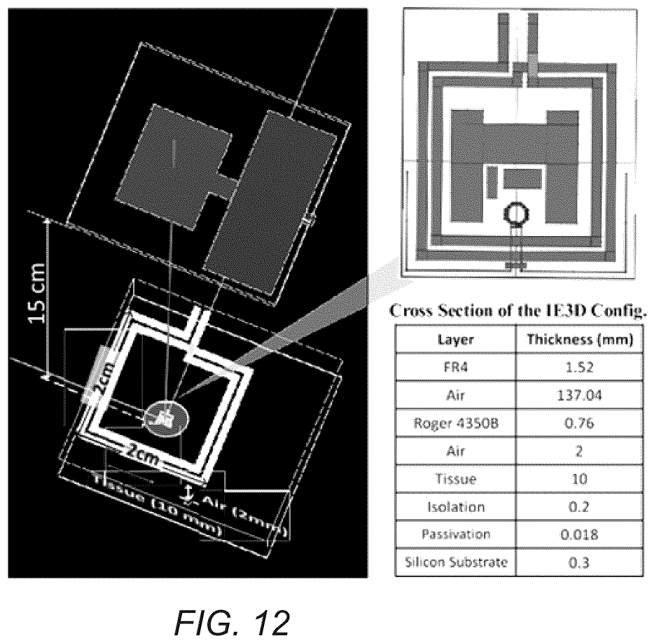

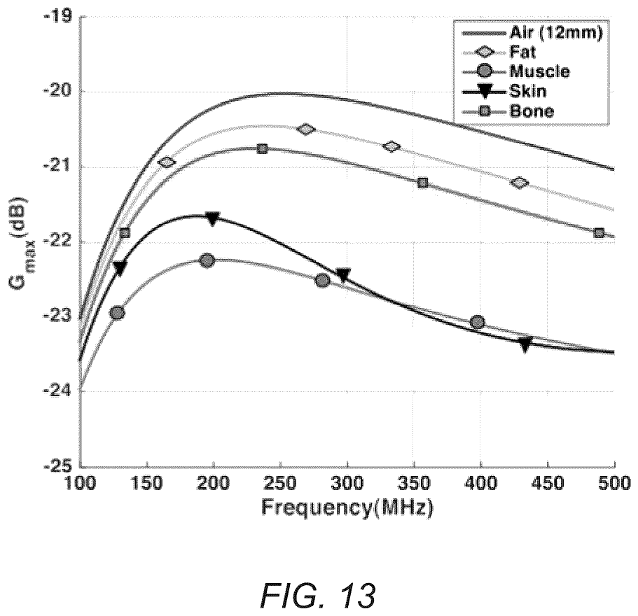

To address the effect of biological tissues and choose the optimal frequency for DL and UL paths, many embodiments can use a simulation tool. A simulation configuration in accordance with an embodiment of the invention is illustrated in FIG. 12. For higher accuracy, a model of the chip including the OCC, the dipole antenna, and metallic parasitics can be used in simulation and the layer map of the design kit can be imported into the simulation tool. Also, the chip can be surrounded by a medical-grade encapsulation with a thickness of 200 μm. The S-parameters of each wireless link can be acquired in the presence of the other link to ensure all coupling effects between UL and DL are taken into effect. The impact of biological tissues can be evaluated by inserting a 10-mm planar layer above the encapsulation layer, which represents various types of biological tissues. Using the S-parameters from EM simulations, the maximum power-transfer efficiency of the WPT system for different link com-positions is plotted and reported in FIG. 13. The optimum frequency and the link efficiency can be both dependent on the link composition.

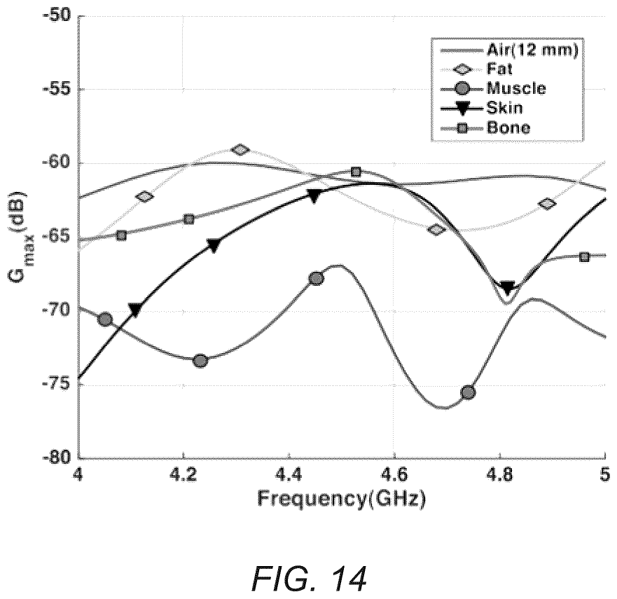

The link efficiency and optimal operating frequency for the UL path can also be acquired from simulations. Considering the area limitation for implementing the TX circuitry, it may be desired to increase the center frequency of the UL communication. On the other hand, at higher frequencies, tissue attenuation can outweigh the advantages of TX circuitry and degrades the overall performance. A maximum link efficiency of the UL path for various tissue compositions in accordance with an embodiment of the invention is illustrated in FIG. 14 Simulation results show the efficiency of the UL two-port network, composed of the monopole and dipole antennas, shows a near-flat response in the frequency range between 4 and 5 GHz for different tissue types in the configuration. Therefore, the center frequency of the TX block can be chosen to be in the middle of this range.

In many embodiments, the maximum link efficiency can be achievable under conjugate impedance matching. Ideally, the frequency dependence of the circuit blocks connecting to the dipole antenna and the OCC should be considered in frequency selection. However, the frequency dependence of such blocks can be much less than the wireless link. Hence, the performance dependence of circuit blocks following the EM structures can be excluded from the optimal frequency selection. However, the power loss due to the impedance mismatch at UL and DL can be considered.

### Circuit Implementation

### Power Management Unit

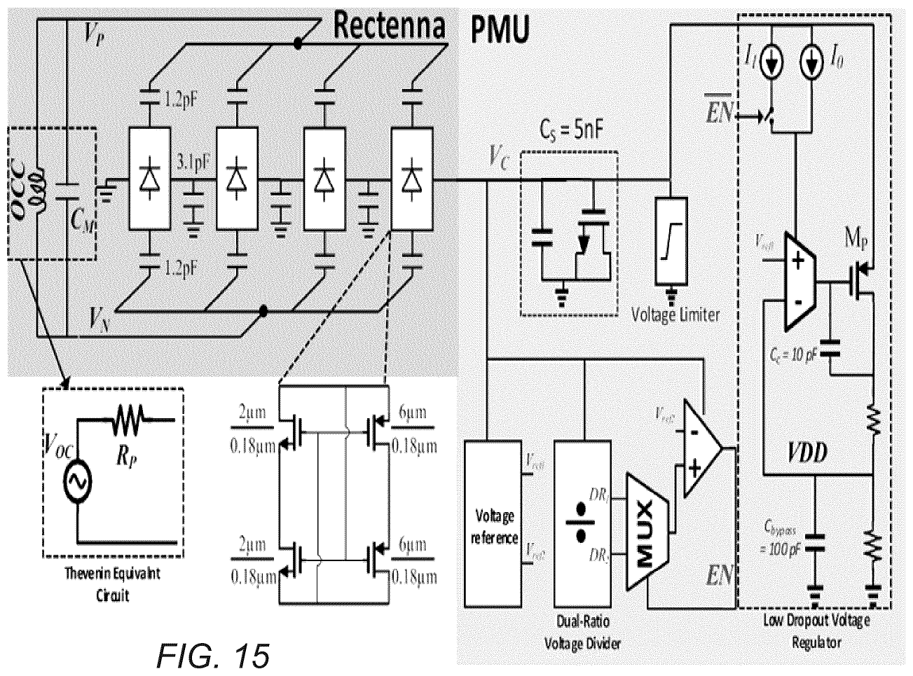

A circuit architecture of a power-harvesting system in accordance with an embodiment of the invention is illustrated in FIG. 15. The rectenna can be implemented with a four-stage full-wave rectifier to ensure V_{C} reaches the required voltage level for the proper operation of the PMU when the transmitted power of the external coil is kept below safety limits. Depending on the received power, and the Q-factor of the OCC and the matching network, several architectures can be used for implementing a voltage rectifier. Diode-connected MOS devices, native MOS, threshold-compensated, and self-driven rectifiers can be named as some examples. Among various topologies, self-driven rectifiers with cross-coupled CMOS devices are known for providing a good balance between conversion efficiency and sensitivity. Hence, many embodiments of the system can select this configuration for implementing a multistage voltage rectifier. To maximize rectifier RF-dc conversion efficiency, transistors dimensions can be optimized. Moreover, deep N-Well NMOS transistors can be used to allow a direct connection between bulk and source terminals. The direct bulk-to-source connection eliminates body effect and prevents NMOS devices' threshold voltage increasing rendering to the RF-dc conversion efficiency improvement. A first-order matching circuit can be realized using a shunt capacitor that resonates with the OCC and the voltage rectifier at the operating frequency. The shunt capacitor cancels out the imaginary part of impedance values. Hence, the power reflection between the OCC and the rectifier can be attributed to the difference in the real-part of their impedances. An equivalent circuit model for the OCC is illustrated in FIG. 15 where the OCC is modeled as a source with an open circuit voltage of Voc and an internal resistance of Rocc. At 250 MHz, EM simulation results show the Rocc as 305 Q. On the other hand, during the sleep mode, the voltage rectifier periodically charges the C_{S} from V_{L} to V_{H}. Depending on the charging speed, the load of the rectifier varies between 235 and 420 μW and the simulated conversion efficiency at 250 MHz varies between 30% 65%. Also, the large signal S-parameter (LSSP) simulation of the rectifier indicates the insertion loss between the OCC and the voltage rectifier is about 4.2 dB. Hence, the overall power-transfer efficiency from the external coil to the rectifier is 24.2 dB. On the other hand, the sensitivity of the power-harvesting system can be defined as the minimum required power transmitted from the external coil to establish a hysteresis operation in the PMU. Based on the simulation results, the sensitivity of the power-harvesting system is 21.5 dBm.

In many embodiments, a behavior of the PMU in the duty cycled mode resembles a hysterics comparator that is realized using a voltage divider, a multilevel reference generator, a MUX, and a voltage comparator as illustrated in FIG. 15 in accordance with an embodiment of the invention. The voltage reference block can be realized with a supply independent proportional-to-absolute-temperature (PTAT) architecture to generate two reference voltages. The voltage divider, MUX, and the comparator can be designed with a particular circuit schematic. Once the TX block is activated, C_{S} discharges and V_{C} drops rapidly. The t_{discharge} is proportional to the value of C_{S}. Therefore, it can be desired to maximize the capacitance value of C_{S} to extend the active mode. To achieve a high capacitance value with area constraints of an on-chip design, many embodiments of the system can stack MIM capacitors over MOSCAP devices with a density of 2 and 5.5 fF/μm², respectively, to realize a 5-nF capacitor.

In many embodiments, a low-dropout (LDO) voltage regulator is incorporated into the PMU to provide a constant 1.3-V dc voltage for the operation of the TX and RX blocks. During the sleep mode, the total current consumed by the LDO is 10 μA. The transition from sleep mode to the active mode can cause a significant load variation for the LDO. On the other hand, a small quiescent current consumption can limit the transient response of the LDO. Hence, the abrupt variation of the load may lead to a large voltage variation at the output of the LDO. The maximum instantaneous current drawn by the TX block can reach as high as 4.5 mA and it may result in a maximum transient voltage variation of 175 mV. To ensure that the LDO remains functional, the bandwidth of the error amplifier can be increased at the onset of the active mode. The PMU in accordance with many embodiments of the system adaptively increases the bias current of the error amplifier by 100 μA resulting in enabling the LDO to maintain the voltage variation below 12 mV. It is ensured the LDO stability conditions are met during the operation. Simulation results show the minimum phase margin of the LDO is 88° and the gain marginal ways remains above 20.5 dB. Although FIG. 15 illustrates a particular circuit schematic of a power-harvesting system, any of a variety of circuit architectures can be utilized as appropriate to the requirements of a specific application in accordance with various embodiments of the invention.

### Data RX Circuit Implementation

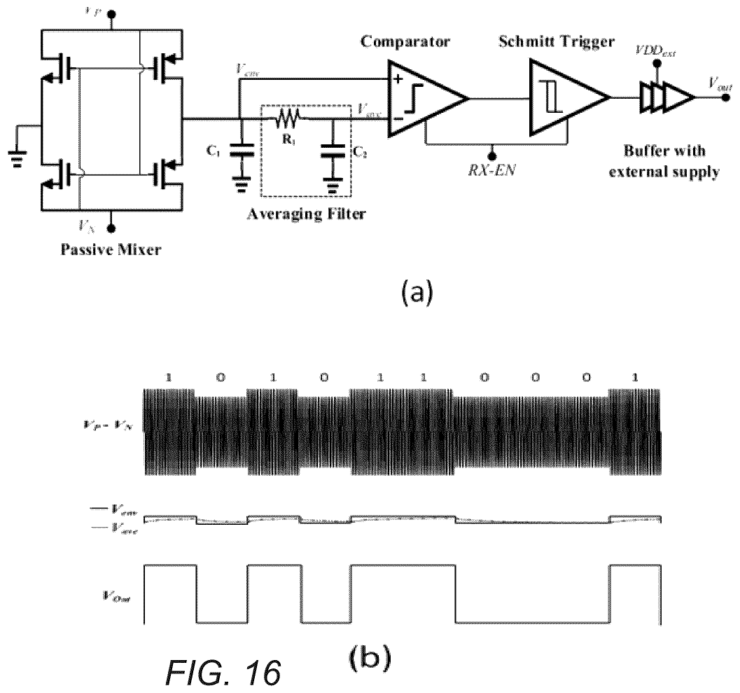

A circuit schematic and timing diagram of a data RX in accordance with an embodiment of the invention ill are illustrated in FIG. 16. The RX can be implemented based on a self-mixing architecture. The power carrier can be modulated with an ASK modulation scheme to carry the DL data stream to the chip. The received RF signal by the OCC can be formulated as

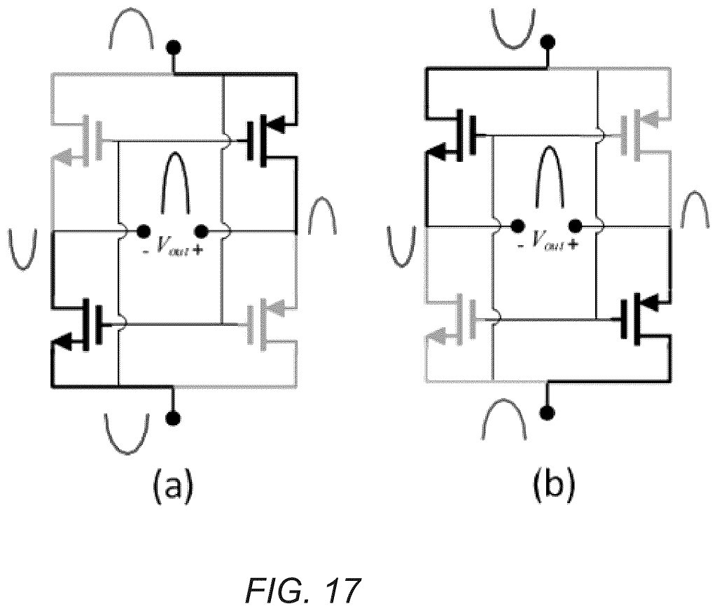

V_{RF}(t)=V_{P}(t)−V_{N}(t)=A cos(2πf_{RF}t)[1+mx(t)] (1)

where x(t) and m represent DL data and modulation index, respectively. Data modulation should have a minimal impact on power flow to the chip. Hence, the RX should be able to detect the DL signal with a very small modulation index. A passive mixer can be implemented using the same circuitry as a single rectifier stage to minimize the power consumption of the RX. Inspecting the switching status of the transistors in positive and negative cycles of V_{RF}, shown in FIG. 17, reveals a single rectifier stage can act as a self-mixer. The behavior of the self-mixer can be approximated as the multiplication of a square wave with a frequency of f_{RF} with V_{RF}. Considering only the first harmonic of the square wave, the output of the mixer can be approximated as

$$\begin{matrix}{V}_{out} = {V}_{RF} \times \frac{4}{π} {\sum }_{n = 1 , 3 , 5 , …}^{∞} cos ( n \times 2 π {f}_{RF} t ) \\ \approx {V}_{RF} \times \frac{4}{π} cos ( 2 π {f}_{RF} t ) \\ = \frac{2 A}{π} [ 1 + mx ( t ) ] ( 1 + cos ( 4 π {f}_{RF} t ) ) .\end{matrix} \qquad ( 2 )$$

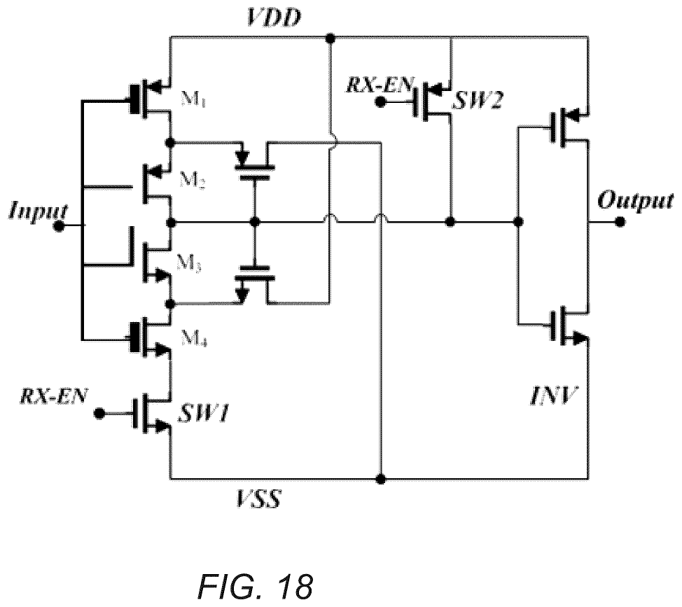

The required data rate for DL communication usually is a few Mbps. Hence, the frequency of x(t) is considerably lower than f_{RF}. To extract DL data, the output of the mixer should be passed from a bandpass filter (BPF) to remove frequency components at dc and 2 f_{RF} whereabouts. The minimum required voltage amplitude for activating the voltage rectifier in the PMU can be 650 mV. Hence, V_{RF} can be directly passed to the mixer with no preamplification. Considering the frequency of the DL data, realizing a BPF with on-chip components may not be realistic. Hence, the BPF in accordance with many embodiments of the system can be implemented in three steps using low-pass filters (LPFs) and a voltage comparator. The output node of the mixer in FIG. 16 is connected to a 10-pF shunt capacitor, which forms an LPF with the output resistance of the mixer. The LPF extracts the envelope of V_{RF}, which consists of a dc term and x(t) according to equation (1). Next, Vₑₙᵥ can be passed through an LPF that has a cutoff frequency of 160 Hz. Due to the large time constant of the LPF, it acts as an averaging filter and the transition time of Vₐᵥₑ is considerably larger than Vₑₙᵥ. To remove the dc component and retrieve x(t), Vₑₙᵥ and Vₐᵥₑ are passed to the voltage comparator. The simulated small-signal gain of the comparator is 51 dB with a 3-dB bandwidth of 3.8 MHz and current consumption of 280 nA. As illustrated in the timing diagram illustrated in FIG. 16, if x(t) does not toggle for multiple consecutive periods, Vₐᵥₑ becomes closer to Vₑₙᵥ. As a result, the comparator can be prone to metastability. To ensure the fidelity of the recovered data is preserved, the comparator can be followed by a Schmitt trigger that introduces a hysteresis effect. Hence, Vout can become insensitive to the voltage variation of the comparator in the metastable mode. The hysteresis effect can also reduce the noise sensitivity of the RX block. A circuit schematic of a Schmitt trigger in accordance with an embodiment of the invention is illustrated in FIG. 18. In many embodiments, transistors are sized properly to achieve a hysteresis window from 385 to 935 mV. The output of the Schmitt trigger can be passed to an on-chip buffer that is powered with an external supply in order to enable a direct connection to a voltage oscilloscope for measurement purposes. Although FIG. 18 illustrates a particular circuit schematic of a Schmitt Trigger in the RX, any of a variety of circuit architectures can be utilized as appropriate to the requirements of a specific application in accordance with various embodiments of the invention.

### Data TX Circuit Implementations

In many embodiments, data communication can be the most power-consuming task in the system. Hence, the TX block can be realized with minimal complexity to reduce the overall power consumption. Due to the amplitude-based modulation, there may be no need for generating an accurate frequency. Therefore, the process, voltage, and technology variations can be tolerated, which can significantly relax the constraints on a TX circuit design. As a result, a free-running oscillator can be adequate to generate a GHz-range carrier frequency for the TX. Data TXs based on amplitude modulations have been reported previously where an LC oscillator is followed by a power amplifier (PA) to drive the antenna. However, the total power consumption of such TXs is above 10 mW resulting in a limited data rate and energy efficiency. To reduce the power consumption of the TX, many embodiments of the system have implemented a PO as the core of the TX block. In many embodiments, the PO can be directly connected to the dipole antenna and drive it without the need for any extra power consumption in buffers. Hence, many embodiments of the system can adopt a co-design approach for the PO and the dipole antenna to set the resonance frequency and maximize the dc-RF efficiency of the TX.

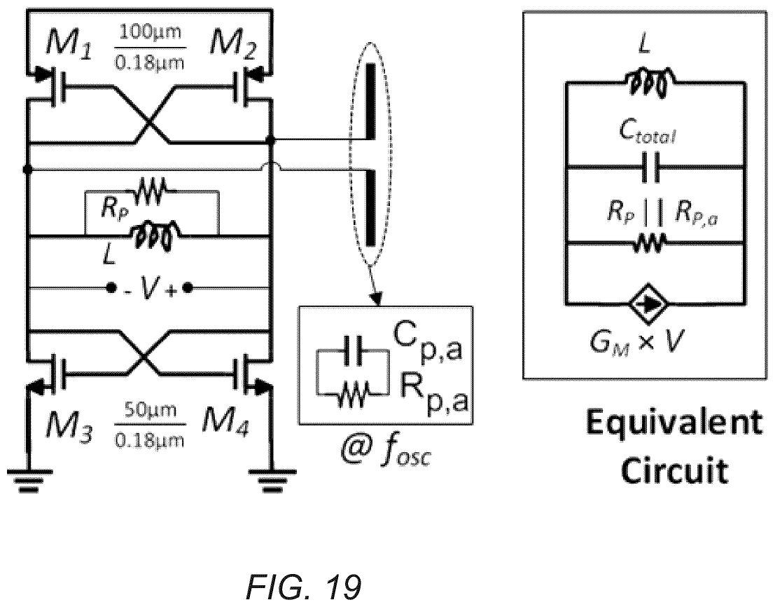

In many embodiments, at the resonance frequency, the antenna can be modeled by a parallel resistor (R_{p,a}) and a shunt capacitor (C_{p,a}). An equivalent model for the PO is demonstrated alongside its circuit schematic in accordance with an embodiment of the invention is illustrate in FIG. 19. The PO can be realized with a class-D topology where the tail transistor is removed resulting in the elimination of the overhead voltage. Unlike conventional oscillators, transistors in class-D can operate as close-to-ideal switches and thus M₁-M₄ are sized properly to guarantee a small on-resistance. Because of the high oscillation amplitude, this structure can be popular for low-phase-noise and low-power applications. The product of current through MOS switches and the supply voltage may be negligible across the oscillation period and the class-D can achieve an energy efficiency of 90%. The power consumption of the PO can be dominantly determined by the parasitic resistance of the tank inductor. Operating at high frequencies and using a large tank inductor with a better Q-factor can be desirable to minimize the power consumption of the PO. However, wireless link simulation results indicate the UL efficiency may be degraded rapidly at frequencies higher than 5 GHz. Thus, the PO in accordance with many embodiments of the system can designed for a center frequency of 4.2 GHz. To ensure the oscillation happening in the PO, the effective transconductance of the complementary switches should overcome the losses of the inductor and the dipole antenna. The startup condition of the oscillator is expressed in the following equation:

$${G}_{M} > \frac{1}{{R}_{F}} + \qquad ( 3 )$$

To maximize the effective transconductance during the oscillation period (G_{M}), a complementary cross-coupled pair can be utilized to boost the G_{M} through a current reusing technique. Also, transistors M₁-M₄ can be properly sized to ensure the oscillation conditions are met across many technology corners. Although switching performance and G_{M} of M₁ M₄ improves as they become larger, the parasitic capacitance associated with them may also increase with size and reduces the PO resonance frequency. Hence, the transistors are sized as illustrated in FIG. 19 in accordance with an embodiment of the invention.

The impact of surrounding tissues on the PO can be evaluated by EM simulation of the dipole antenna and the tank inductor using stimulation tools.

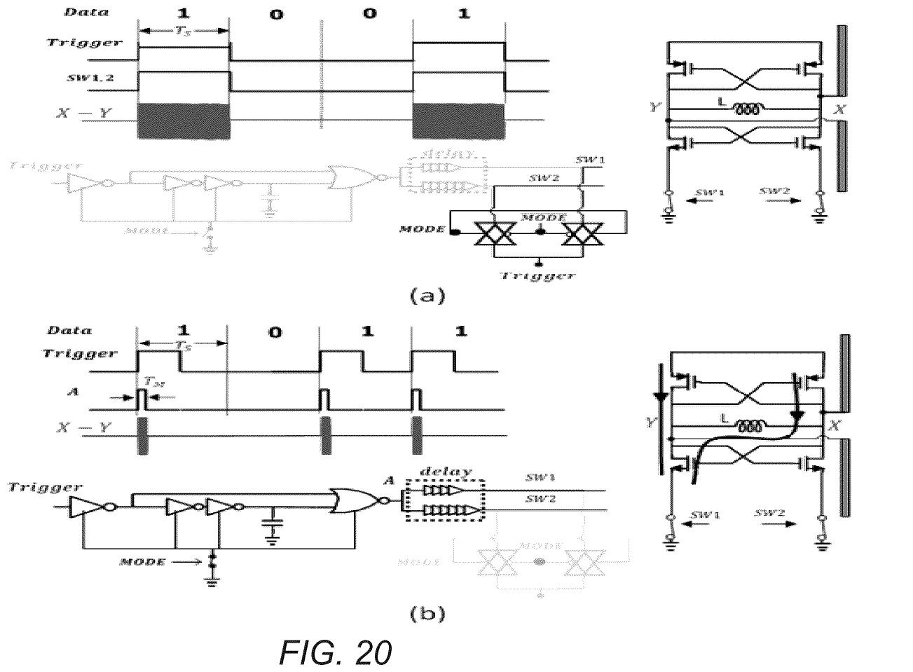

An advantage of implementing the TRX with an FDD scheme is the minimal impact of the power link on the TX operation. To verify this, simulations are provided regarding the coupling efficiency between the external power TX and the dipole antenna connected to the tank inductor. Assuming a power carrier of 250 MHz, the seventeenth harmonic of the power carrier (4250 MHz) is the closest harmonic to the free-running frequency of the PO. The undesired coupling efficiency between the power carrier and the PO is 63.4 dB, 83.7 dB at 250 and 4250 MHz, respectively. Due to the large isolation, the power link does not affect the PO operation and no frequency deviation from the free-running frequency is observed in the TX block. The TX block can be configured to conduct the UL communication with either OOK or UWB modulation scheme. The circuit schematic of a reconfigurable TX and the corresponding waveforms in both operating modes in accordance with an embodiment of the invention is illustrated in FIG. 20. The trigger signal for enabling the PO can be shaped and fed to the TX block according to the modulation type. When operating in OOK mode, the trigger signal replicates the data pattern, whereas it is shaped as a return-to-zero (RZ) waveform for symbol “1” in UWB mode as shown in FIG. 20 item (b). The operating mode of the TX can be controlled by an external mode selection signal that alters the signal path from the trigger to the PO. In OOK mode, the trigger signal can be passed to the PO through a pair of transmission gates to control two switches that connect the NMOS cross-coupled pair to the ground. The switches can be realized with NMOS transistors and can be sized 8 larger than the NMOS cross-coupled pair. As illustrated in FIG. 20 item (a), during transmission of a “1” symbol, the PO is active for the entire symbol period (T_{S}) and it results in smooth switching transitions. Consequently, the transmitted signal from the PO occupies less bandwidth and can be detected with a simpler RX. However, the average power consumption of the TX block in OOK mode can be independent of the data rate and is merely determined by the instantaneous power consumption of the PO. The continuous power received by the power-harvesting system can be about a few hundreds of micro-Watt. On the other hand, the power consumption of the TX block can be in the milli-Watt range and it is expected the data communication in the OOK mode to be duty cycled. The TX block can achieve a significantly lower average power consumption in UWB mode at the expense of a larger occupied bandwidth. In UWB mode, the trigger signal can be first passed through a digital circuitry that generates a short impulse (T_{M} is approximately 2 ns) upon the detection of a rising edge in the trigger waveform. Due to the small impulse duration, it may be imperative to ensure the PO can reliably startup. Asymmetric driving of an oscillator is a known technique to achieve a fast startup. Therefore, generated impulse can be delayed by an inverter chain delay line that connects SW2 slightly after SW1. The current flow during the startup time window through the PO, shown with arrow lines in FIG. 20 item (b), creates an initial voltage difference across node X and Y resulting in a startup time of ˜200 ps.

Although specific implementations for a fully integrated SoC for low-power and miniaturized neural recording systems are discussed above with respect to FIGS. 1-20, any of a variety of implementations utilizing the above discussed techniques can be utilized for an Soc in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. An implantable neural interface comprising:
an electrode array having a plurality of electrodes;
front end circuitry comprising:
one or more digital components; and
at least one amplifier coupled to a first electrode and a second electrode of the electrode array, wherein the amplifier and the first electrode and the second electrode form a sensing channel configured to sense electrical activity; and
a transceiver comprising:
a plurality of digital components;
a power harvesting system that receives RF energy through a wireless power link; and
a wireless clock receiver that provides a clock signal to the one or more digital components of the front end circuitry and the plurality of digital components of the transceiver.

2. The implantable neural interface of 1, wherein the at least one amplifier is configured to increase a voltage amplitude of a signal corresponding to the sensed electrical activity.

3. The implantable neural interface of 2, wherein the one or more digital components of the front end circuitry comprise an Analog to Digital Converter (ADC) coupled to the at least one amplifier and configured to convert an amplified signal to a digital signal.

4. The implantable neural interface of 3, wherein:
the transceiver comprises a data transmitter (TX) configured to transmit data corresponding to the sensed electrical activity; and
the one or more digital components of the front end circuitry comprise a Parallel-Input-Serial-Output (P ISO) shift register unit coupled to receive the digital signal from the ADC and configured to output digitized data corresponding to the digital signal to the transceiver.

5. The implantable neural interface of 1, wherein the transceiver further comprises:
a data receiver (RX) for receiving data from an external reader on a wireless downlink (DL);
a reconfigurable data transmitter (TX) for transmitting data to the external reader on a wireless uplink (UL);
a plurality of antennas that enable simultaneous power delivery and data communication through two distinct wireless links separated in the frequency domain; and
a receiver antenna that is shared between the power harvesting system and the RX.

6. The implantable neural interface of 5, wherein the data TX comprises a power oscillator (PO) directly connected to a transmitter antenna for wireless data transmission.

7. The implantable neural interface of 5, wherein DL data is incorporated into the wireless power link with an amplitude-shift-keying (ASK) modulation scheme.

8. The implantable neural interface of 5, wherein simultaneous UL and DL communication is enabled using frequency division duplexing (FDD), wherein a center frequency of the UL is in the GHz region.

9. The implantable neural interface of 5, wherein the data TX is configured to transmit UL data with either on-off-keying (OOK) and ultrawideband (UWB) modulation.

10. The implantable neural interface of 5, wherein the RX is directly powered by the power harvesting system and is active during operation of the neural interface.

11. The implantable neural interface of 5, further comprising a voltage rectifier that co-optimizes the receiver antenna and the wireless data transmission to maximize power-transfer efficiency.

12. The implantable neural interface of 5, wherein the plurality of antennas comprises a dual-antenna architecture that minimizes the interference between the power link and the data TX.

13. The implantable neural interface of 5, wherein the transceiver is placed on top of an analog recording and stimulation front-end (AFE) unit comprising the electrode array.

14. The implantable neural interface of 5, wherein the receiver antenna is on-chip.

15. The implantable neural interface of 5, wherein the transmitter antenna is on-chip.

16. The implantable neural interface of 5, wherein the transceiver is implemented on a single CMOS silicon chip and all components for power delivery, energy storage, data communication, including an antennas, are implemented on the same chip.

17. The implantable neural interface of 5, wherein the receiver antenna is a loop, and the transmitter antenna is a dipole.

18. The implantable neural interface of 5, wherein the receiver and transmitter antenna use different polarizations to maximize isolation.

19. The implantable neural interface of 1, wherein the front end circuitry and the transceiver define a rectangular form factor comprising:
a skull facing side with the transceiver; and
a brain facing side with the front end circuitry and the electrode array.

20. The implantable neural interface of 1, further comprising a housing, wherein:
the plurality of electrodes are associated with an exterior surface of the housing; and
the front end circuitry and the transceiver are associated with an interior of the housing.

21. The implantable neural interface of 1, wherein the electrode array, the front end circuitry, and the transceiver are embodied as a system on a chip (SOC).

22. An implantable medical device comprising:
an electrode array having a plurality of electrodes;
front end circuitry comprising:
one or more digital components; and
at least one amplifier coupled to a first electrode and a second electrode of the electrode array, wherein the amplifier and the first electrode and the second electrode form a sensing channel configured to sense electrical activity; and
a transceiver comprising:
a plurality of digital components;
a power harvesting system that receives RF energy through an inductive wireless link;
a wireless clock receiver that provides a clock signal to the one or more digital components of the front end circuitry and the plurality of digital components of the transceiver; and
a data transmitter configured to transmit data corresponding to the sensed electrical activity.

23. The implantable medical device of claim 22, wherein the electrode array is configured for implant in or on a heart, and the sensed electrical activity corresponds to electrical cardiac activity.

24. The implantable medical device of claim 22, wherein the electrode array is configured for implant in or on a brain, and the sensed electrical activity corresponds to electrical neural activity.

25. The implantable medical device of claim 22, wherein the electrode array is configured for implant in or on a spine, and the sensed electrical activity corresponds to electrical neural activity.

26. A fully integrated system-on-chip (SOC), comprising:
a data transceiver (TRX) that is powered through a radio frequency (RF) power link, comprising:
a power-harvesting system comprising a rectifier and a power management unit (PMU);
a data receiver (RX) for receiving data from an external reader on a wireless downlink (DL);
a reconfigurable data transmitter (TX) for transmitting data to the external reader on a wireless uplink (UL);
a plurality of antennas that enable simultaneous power delivery and data communication through two distinct wireless links separated in the frequency domain;
a receiver antenna that is shared between the power harvesting system and the RX;
wherein the TX comprises a power oscillator (PO) directly connected to a transmitter antenna for wireless data transmission.

27. The SOC of claim 26, wherein DL data is incorporated into the power link with an amplitude-shift-keying (ASK) modulation scheme.

28. The SOC of claim 26, wherein simultaneous UL and DL communication is enabled using frequency division duplexing (FDD), wherein a center frequency of the UL is in the GHz region.

29. The SOC of claim 26, wherein the TX is configured to transmit UL data with either on-off-keying (OOK) and ultrawideband (UWB) modulation.

30. The SOC of claim 26, wherein the RX is directly powered by the power-harvesting system and is active during the entire operation of the system.

31. The SOC of claim 26, further comprising a voltage rectifier that co-optimizes the power receiving antenna and the wireless data transmission to maximize power-transfer efficiency.

32. The SOC of claim 31, wherein the PMU converts unregulated output voltage of the rectifier to a constant dc voltage and adjusts the power consumption of the system.

33. The SOC of claim 26, wherein the PMU sets the operating mode and biasing condition for different components of the TRX based on their power consumption and the total available power budget.

34. The SOC of claim 26, wherein, depending on the power consumption of each block, the PMU sets its power delivery scheme to either continuous or duty cycled.

35. The SOC of claim 31, further comprising a storage capacitor (Cₛ) that stores converted energy by the rectifier and a voltage limiter is included in the PMU to prevent voltage breakdown, wherein the PMU monitors the voltage level across Cₛ and establishes active and sleep modes for the TX operation.

36. The SOC of claim 26, wherein the plurality of antennas comprises a dual-antenna architecture that minimizes the interference between the power link and the TX.

37. The SOC of claim 26, wherein the TRX uses an amplitude-based modulation scheme to maximize energy efficiency.

38. The SOC of claim 26, wherein the TRX is placed on top of an analog recording and stimulation front-end (AFE) unit with a 2D microelectrode array comprising a plurality of electrodes.

39. The SOC of claim 26, wherein the TRX acts as a communication hub between electrodes within an AFE unit and the external reader.

40. The SOC of claim 26, wherein the receiver antenna is on-chip.

41. The SOC of claim 26, wherein the transmitter antenna is on-chip.

42. The SOC of claim 26, wherein the TRX is implemented on a single CMOS silicon chip and all components for power delivery, energy storage, data communication, including an antennas, are implemented on the same chip.

43. The SOC of claim 26, wherein the receiver antenna is a loop antenna, and the transmitter antenna is a dipole antenna.

44. The SOC of claim 26, wherein the receiver and transmitter antenna use different polarizations to maximize isolation

45. A fully integrated system-on-chip (Soc) for neural stimulation and recording, comprising:
a power harvesting system that receives RF energy through an inductive wireless link;
a wireless clock receiver that provides a clock signal to a plurality of digital components;
an amplifier that senses neural activity and increases a voltage amplitude of a signal.

46. The system-on-chip of claim 45, further comprising an Analog to Digital Converter (ADC) that converts an amplified signal to a digital signal.

47. The system-on-chip of claim 46, wherein the ADC is a Successive Approximation Register (SAR) ADC.

48. The system-on-chip of claim 45, further comprising a Parallel-Input-Serial-Output (PISO) shift register unit that loads digitized data from the recording channels and passes the serialized data stream to the data TX.

49. The system-on-chip of claim 48, wherein the data TX is an ultra-wideband (UWB) transmitter operating in the 3-10 GHz range.

50. The system-on-chip of claim 48, wherein the data TX operates in the ISM bands between 10 MHz and 10 GHz.

51. The system-on-chip of claim 48, wherein the data TX uses at least modulation scheme selected from the group consisting of on-off-keying (OOK), amplitude-shift-keying (ASK), and UWB modulation.

52. The system-on-chip of claim 45, wherein an operating mode of the transmitter is controlled by an external mode selection signal.

53. The system-on-chip of claim 45, wherein control commands provide information on the start and end time of the neural sensing, clock rate, number of bits for digitization, the frequency of the transmitter, and the modulation of the transmitter signal.

54. The system-on-chip of claim 53, wherein the controlled commands are generated by an external device and transmitted wirelessly to the SOC.

55. The system-on-chip of claim 45, wherein the voltage waveforms of heart are detected and amplified.

56. The system-on-chip of claim 45, wherein the voltage waveforms of an implantable sensor is measured and amplified.
